(19) United States
(12) Patent Application Publication
(10) Pub. No.: US 2015/0125136 A1
(43) Pub. Date: May 7, 2015

(54) Vaporization & Inhalation System with Convective and Conductive Heaters for Herbal Material and/or Fluid Extracts

(71) Applicant: Timothy H. Sanchez, Somerville, MA (US)

(72) Inventor: Timothy H. Sanchez, Somerville, MA (US)

(21) Appl. No.: 14/532,720

(22) Filed: Nov. 4, 2014

Related U.S. Application Data
(60) Provisional application No. 61/899,920, filed on Nov. 5, 2013.

(51) Int. Cl. A61L 9/03

(52) U.S. Cl. CPC A61L 9/03
USPC 392/394; 392/386

(57) ABSTRACT

Disclosed is an appliance for volatilizing active substances in either herbal material or fluid extract for inhalation, including a material vaporization surface with a plurality of convective air flow apertures, a conductive heater and a convective heater.

### CROSS REFERENCE TO RELATED APPLICATION

This application claims the benefit of U.S. Provisional Patent Application Ser. No. 61/899,920, filed Nov. 5, 2013 (EFS ID: 17313604), which this application also hereby incorporates herein by this reference and claims the priority of.

### TECHNICAL FIELD

This invention pertains to a vaporization system for use in the field of active substance volatilization for aroma therapeutic purposes, and more particularly, to such a system which may provide both convective heat and conductive heat to vaporize material or substance in herbal or liquid form.

### BACKGROUND OF THE INVENTION

For several years alternatives to burning or combusting herbal material and/or fluid material have been sought to avoid the negative health effects from inhaling the products of combustion. Due to the different nature of vaporizing substances from solid herbal material compared to vaporizing liquids and resins, the prior art has independently pursued the vaporization.

There are prior art devices that: are solely directed to vaporizing active substances through convective heating which generally volatilize the active substances in herbal material by passing hot air through the material; and other devices that are directed to vaporizing active substances solely through conductive heating. The vaporization through conductive heating generally involves the heating of the fluid, oil or extract material by directly or indirectly placing it on a conductive heating element.

Whether through convective heating or conductive heating, the heat volatilizes the targeted compound in a liquid or solid form or phase, and changes or converts the phase to a gaseous phase or vapor that can then be inhaled or contained for later inhalation or use.

The use of hot air via the convective heating is preferred for extracting the targeted substance from herbal material as it is more efficiently or smoothly extracted with the hot air passing through and around it. When put directly in contact with a conductive heater, non-uniform heating or burning of the herbal material is more likely to occur. On the other hand, the use of conductive heat is generally preferred for the volatizing or vaporization of the targeted substance contained within a fluid or oil extract material.

On the other hand when the targeted substance is contained within a fluid or oil extract, the substance is more efficiently vaporized through the utilization of a conductive heater. Convective heaters generally take unnecessarily long amounts of time to vaporize oil or fluid, due in part to the small surface area contact between the hot air and the fluid extract.

Users who vaporize material in both herbal and liquid form are therefore in a position where they must buy two different and relatively expensive vaporizers, or settle for inefficient or undesirable vaporization of either their herbal material or their liquids, extracts and oils.

Despite the longstanding and recognized need to vaporize or volatilize the targeted substance efficiently from both herbal and liquid material, there are no acceptable alternative systems or devices for accomplishing this, including with both a convective heater and a conductive heater.

The present invention circumvents the limitations on current vaporizer/inhaler systems having developed a way to achieve the benefits of both in one system or device. Embodiments of this invention achieve this through the use of a dual-heating system, which includes both a convective heating cartridge (a hot air generator) to provide hot air flow and a conductive heating plate directly in the receptacle that holds herb and fluid.

The utilization of two types of heaters provides the capacity to vaporize the targeted substance from both herbal material and from fluid more efficiently. Embodiments of this system further include ways to optimize the vaporization process for extracting the targeted substance from both types of material (herbal and fluid) by also providing the ability to combine or concurrently provide heat from both types of heaters to achieve higher and more desirable efficiencies. Embodiments of this invention further include the ability to operate in two different modes for either herbal material and/or fluid material, to optimize the vaporization process.

For example embodiments of this invention may provide a control system which may utilize the conductive heat source to pre-heat the herbal material to a given temperature, such as 120 degrees F., and then introduce the convective air flow through the herbal material, to more evenly volatilize the targeted substance with reduced vaporization time.

It is therefore an object of some embodiments of this invention to provide a dual heater vaporization system which uniquely provides a solution which allows the user to optimize the vaporization of the targeted substance, whether from an herbal material or from a liquid, oil or extract. Embodiments of this invention accomplish this by providing for the use of the conductive heating source alone, the use of the convective heating source alone, or combinations of both the conductive heating source and the convective heating source.

Other objects, features, and advantages of this invention will appear from the specification, claims, and accompanying drawings which form a part hereof. In carrying out the objects of this invention, it is to be understood that its essential features are susceptible to change in design and structural arrangement, with only one practical and preferred embodiment being illustrated in the accompanying drawings, as required.

### BRIEF DESCRIPTION OF THE DRAWINGS

Preferred embodiments of the invention are described below with reference to the following accompanying drawings.

FIG. 1 is a front perspective view of one example of an embodiment of this invention, wherein the housing is partially transparent;

FIG. 2 is a front perspective exploded view of the example of the embodiment of the invention illustrated in FIG. 1;

FIG. 3 is a perspective cross-section view of an example of a convective heating unit and column which may be utilized in embodiments of this invention;

FIG. 4 is a perspective view of an example of a convective heating column with a bowl unit clipped to the heating column;

FIG. 5 is a perspective transparent view of an example of a bowl unit and conductive heater which may be utilized in embodiments of this invention;

FIG. 6 is an elevation perspective exploded view of an example of a bowl unit, material vaporization surface and conductive heater combination that may be utilized in embodiments of this invention;

FIG. 7 is a perspective view of an example of a container filling adapter or head which may be utilized in embodiments of this invention;

FIG. 8 is an example of one flowchart of control and electronic functions for the herb material mode, that may be utilized in embodiments of this invention;

FIG. 9 is an elevation cross-sectional view of an example of an embodiment in which a hose is attached as a conduit for a user to draw or inhale the air containing the vaporized material;

FIG. 10 is a top view of an embodiment of the conduction heater and material vaporization surface; and

FIG. 11 is a perspective view of the embodiment of the invention illustrated in FIGS. 1 & 2 with the housing removed.

### DETAILED DESCRIPTION OF THE PREFERRED EMBODIMENTS

Many of the fastening, connection, manufacturing and other means and components utilized in this invention are widely known and used in the field of the invention described, and their exact nature or type is not necessary for an understanding and use of the invention by a person skilled in the art or science; therefore, they will not be discussed in significant detail. Furthermore, the various components shown or described herein for any specific application of this invention can be varied or altered as anticipated by this invention and the practice of a specific application or embodiment of any element may already be widely known or used in the art or by persons skilled in the art or science; therefore, each will not be discussed in significant detail.

The terms “a”, “an”, and “the” as used in the claims herein are used in conformance with long-standing claim drafting practice and not in a limiting way. Unless specifically set forth herein, the terms “a”, “an”, and “the” are not limited to one of such elements, but instead mean “at least one”.

Volatilizing an active substance from herbal material is significantly different from vaporizing fluid extract. In the case of herbal material, the herb is most commonly heated by convection heating (passing hot air through the herbal material) until the material reaches the spontaneous vaporization temperature of the active substance (for example nicotine in tobacco is between 95° C. and 112° C.). In this way, the active substance is extracted without combusting the material. By contrast, fluid extract that already contains an active substance may be vaporized by simply boiling the entire fluid (for example propylene glycol (PG) containing nicotine), and inhaling it. This heat is commonly applied by direct contact with a heating element (conductive heating). Temperature ranges for both processes are in a similar range, typically from 125-170° C., but the manner of applying the heat is very different. Embodiments of the present invention may employ two heaters, a convective heating cartridge 26, and a conductive heating plate 82 that sits directly in the bowl (vaporization chamber), allowing it to efficiently vaporize either herb and/or fluid.

Embodiments of this invention are directed to the volatilization or vaporization of active substances within herbal material, as well as the direct vaporization of fluid extracts containing active substances. There are multiple different additional features and components that may be used in some or all embodiments of this invention, in which provide desirable improvements to performance characteristics to the invention. One such example that may be utilized in some aspects of the invention is the dome shape of the housing, which provides a lower center of gravity and more stable base for the system.

Another aspect of an embodiment of this invention is how the vaporized air is routed and/or utilized. In some embodiments of this invention the air containing the vaporized material is directed from the vaporization chamber toward the exit or exhaust conduit for the air with vaporized material to be used. The airflow may be forced convection through the use of a fan, or it may be prompted by a user sucking the air containing the vaporized substance out of the invention.

The airflow through the exhaust may be directed into a container or bag for later use, or it may be directed through a hose or user conduit so the user can receive the airflow with the vaporized substance directly from the invention, all within the contemplation of embodiments of this invention. In the embodiment where the user is receiving the airflow directly from the invention, a mouthpiece may be utilized and in the embodiment where the airflow is directed into a container, a bag or plastic enclosure (containment bag). In the case of a container, it is preferable to have an adapter to easily connect and disconnect the container.

Embodiments of this invention may also include an internal rechargeable battery, providing the option of portability, and/or a storage compartment molded into the housing for convenient storage of herb, fluid, or accessories, which can frequently get lost.

It should also be noted that while the term convective heater is used herein, it is used in its broadest sense and may include without limitation regular convective heaters, heat sources with a forced convection feature attached or detached, or a hot air generator, all within the contemplation of embodiments of this invention.

High usage parts of the device, including the bowl, conductive heating plate, bowl cap, and bag, are separate replaceable components and configured in a modular fashion, which allows for the ability to replace the parts with ease in the event of a part failure. For example, the material vaporization surface may be integral with coils but may also be provided with an inexpensive material so if it needs to be replaced it can be replaced without having to replace other adjacent parts.

REFERENCE NUMBERS IN THE DRAWINGS

| | | | |
|---|---|---|---|
| 10 | Dual-heater vaporizer | 12 | Housing Shell |
| 14 | Bag | 16 | Bag Mouth Piece |
| 18 | Filling Head | 20 | Bowl (containing conductive |
| | | | heater) |
| 22 | Screen | 24 | Housing Shell Side Door |
| 26 | Convective Heating Column | 28 | LCD |
| 30 | Control Panel | 32 | Base |
| 34 | Silicon Base Pad | 36 | Printed Circuit Board (PCB) |
| 38 | PCB Platform Housing | 40 | Fan |
| 42 | Battery | 44 | Battery Harness |
| 46 | Air vents | | |

Convective Heating Column Sub-Parts

| | | | |
|---|---|---|---|
| 48 | Enclosure A (fan side) | 50 | Enclosure B |
| 52 | Electrodes for conductive heater | 54 | Insulating Fabric |
| 56 | Steel Tube | 58 | Convective Heater Cartridge |
| 60 | Steel Mesh Cartridge Cover | 62 | Bowl Ledge |
| 64 | Smart Clips for Bowl | 66 | Key Ridge for Bowl |
| 68 | Wire for bowl | | |

Conductive Heating Bowl Sub-Parts

| | | | |
|---|---|---|---|
| 70 | Outer bowl shell | 72 | Bowl smart clips |
| 74 | Bowl Steel Inner | 76 | Bowl-cartridge Adapter Plate |
| 78 | Bowl Electrodes | 80 | Silicon Electrode Holder |
| 82 | Diffusing Conductive Heater | 84 | Conductive Nicrome Coil |
| 86 | Air Diffusing Holes | 88 | Screw clamps for filling head |
| 90 | Indentation for Oil/Extract | 92 | Lock for column attachment |

Control Panel Buttons

| | | | |
|---|---|---|---|
| 94 | ‘Herb Mode’ On/off button | 96 | Directional pad (up, down, |
| | | | left, right) |
| 98 | ‘Extract Mode’ On/off button | | |

Filling Head

| | | | |
|---|---|---|---|
| 100 | Plastic outer cap | 102 | Inner steel chamber |

The present invention is a compact, stable, and efficient appliance for volatizing active substances in both herbal material, as well as fluid extract.

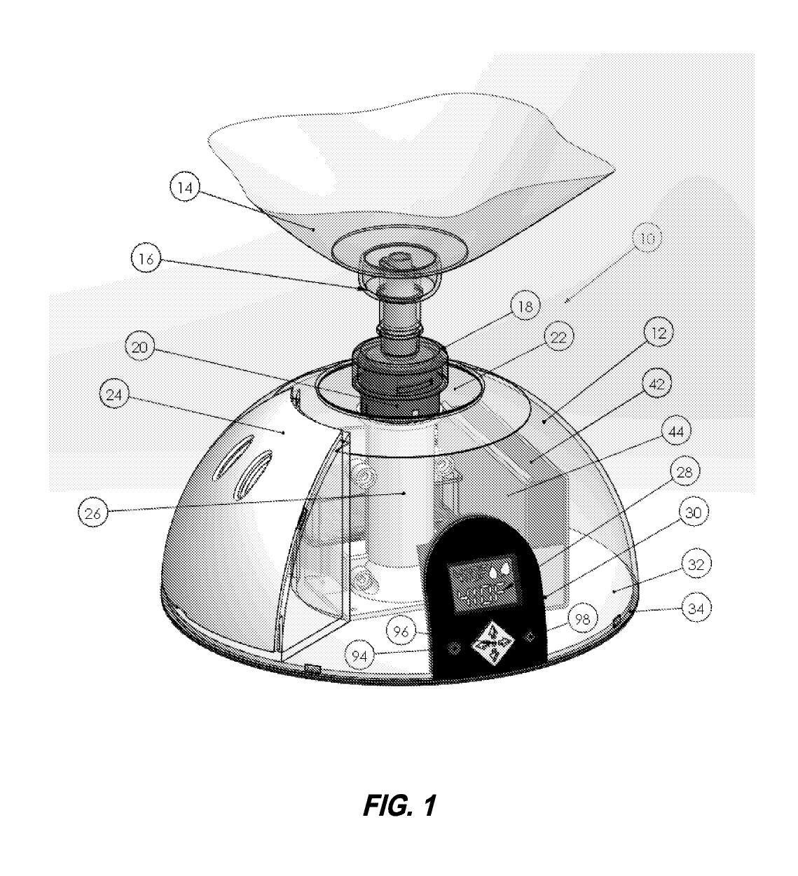

FIG. 1 is a front perspective view of one example of an embodiment of this dual heater vaporizer invention 10, wherein the housing 12 or outer shell is shown as partially transparent so the user may view the internal elements through the outer housing. FIG. 1 illustrates collection bag 14, filling head 18 and bowl 20, wherein the bowl may be a vaporization chamber which includes the material vaporization surface and the conductive heater components. FIG. 1 further illustrates a side door 24 in the housing, convective heating column 26, LCD temperature display 28 for the control panel 30, battery 42, printed circuit board 36, air vents 46, battery harness 44, silicone base pad 34 and base 32.

FIG. 1 also shows the control pad control unit, including an on-off button for herb mode 94, directional pad 96 and extract mode or liquid mode on-off button 98.

FIG. 1 shows an embodiment in which both herbal material and fluid material may be positioned by the user in the bowl 20. The device applies heat to the herb/fluid until the active substances are vaporized. The vapor is transported via air flow through a closed air path, either into a collection bag 14, or for direct inhalation through a mouthpiece and/or hose.

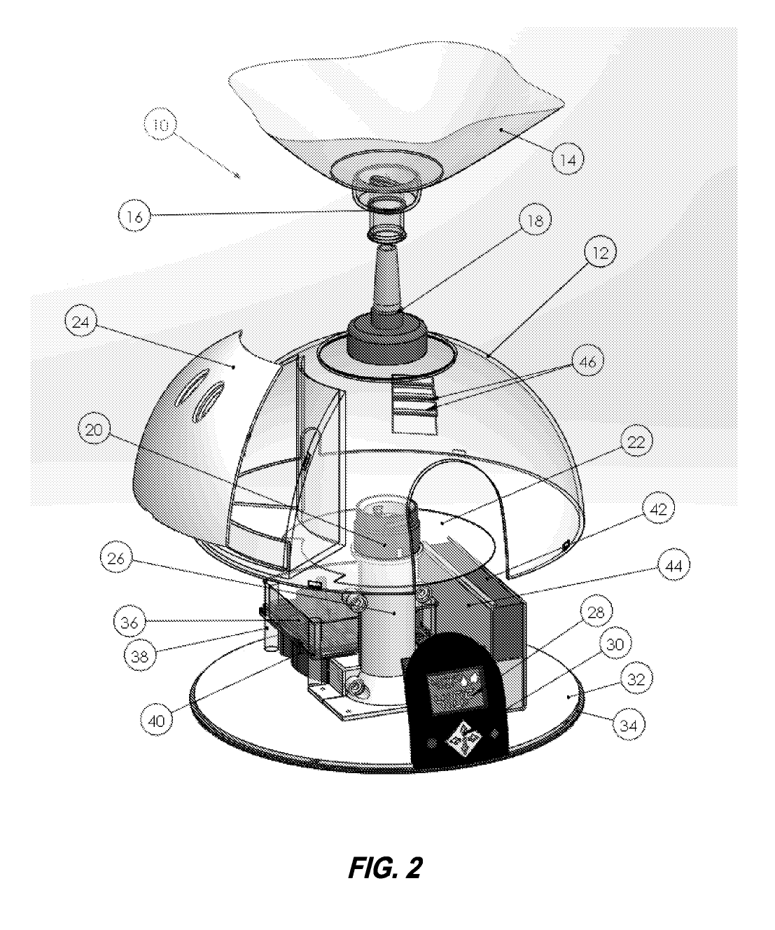

FIG. 2 is a front perspective exploded view of the example of the embodiment of the dual heater vaporizer invention 10 illustrated in FIG. 1, showing collection bag 14, mouthpiece 16 for collection bag 14, filling head 18, housing 12, battery 42 battery harness 44, air vents 46, housing door 24, vaporization chamber or bowl 20, base pad 34, printed circuit board 36, base 32, fan 40, control panel 30 and LED display 28 on control panel 30. FIG. 2 further shows printed circuit board (PCB) platform housing 38 and screen 22.

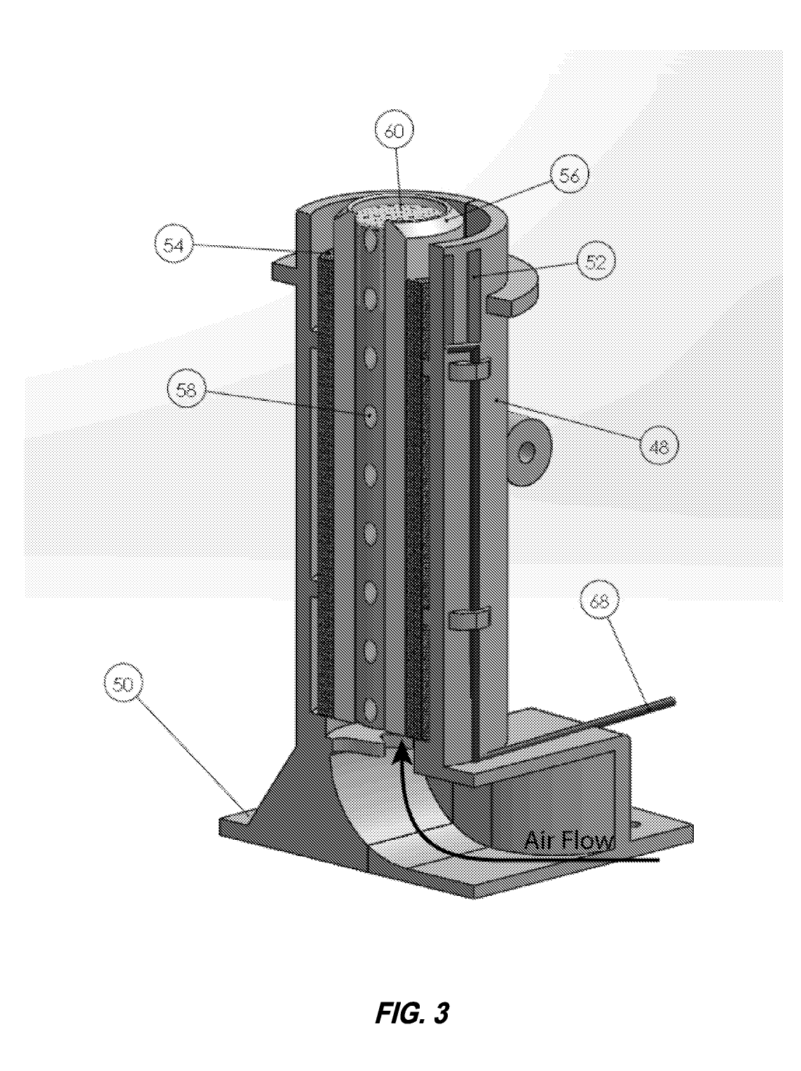

FIG. 3 is a perspective cross-section view of an example of a convective heating unit and column which may be utilized in embodiments of this invention. FIG. 3 illustrates enclosure 50, convective heater cartridge 58, insulating fabric 54, steel mesh cartridge cover 60, steel tube 56, electrodes for conductive heater 52, enclosure 48 on fan side and wire 68 for the bowl.

In embodiments of this invention, whether vaporizing herbal material or fluid, the same path or conduit for air flow may be utilized. As illustrated in FIG. 3, an air flow conduit is provided which allows air to be drawn into the lower portion or base of the convective heating column 26. Air can either be drawn into the chamber by suction if the user is sucking on a hose at the end of the air path (actively drawn convection) or it can be blown through the column by the fan 40 (forced convection), which is attached at or near the bottom opening of the convective heating column enclosure 48. This attachment is preferably sealed or air tight.

After passing through the heating column, air is directed up into the bowl 20 to heat the herbal material on the material vaporization surface. Again, the bowl is preferably tightly attached to the heating column to prevent any air leakage. After the air flows through and receives vaporized material from the vaporization chamber, the air then flows through the filling head 18 (also tightly attached to the bowl) into the containment bag or into the hose if the hose is being utilized.

Air flow is thus contained within a series of closed chambers where hot air is only in contact with steel. This is to prevent any potential health risks of inhaling hot air that had been in contact with plastic.

FIG. 3 is focused on the convective heating column 26, using a section view to visualize the internal components. A convection heater cartridge which may be porous, but otherwise conventional heating cartridge is enclosed in the center of this column. It is immediately surrounded by a length of steel tubing 56 that provides a closed chamber for air to pass through. The top of the tube has a simple chamfer in order to fit tightly into the bottom of the bowl, fitting directly into the bowl-cartridge adapter plate 76. In order to prevent over-heating of the enclosure, a layer of insulating fabric 54 surrounds the steel tube 56. This insulated unit sits within a plastic enclosure consisting of two straight-pull plastic-injection-molded parts. These two parts, enclosure A 48 and enclosure B 50 fit into each other and attach tightly together with screws.

Electrodes 52 are fused to the top of enclosure 48 that are complementary to electrodes on the inner edge of the outer bowl shell 70. These electrodes are for carrying electricity to the diffusing conductive heater 82. When the bowl 20 is attached, the complementary electrodes come in contact, allowing the conductive heater plate 82 to heat up.

It should be noted that while it is preferred to locate the convective heater or hot air generator vertically below the conductive heater or heating element, that is not required to practice embodiments of this invention. Instead, the relative configuration or disposition of the convective heater versus the conductive heater can be in any one of a number of different configurations, such that the conductive heater is directly over and above the convective heater, vertically above (but not directly over) the convective heater, horizontally oriented with respect to one another, or wherein the conductive heater is vertically above the conductive heater, all within the contemplation of embodiments of this invention.

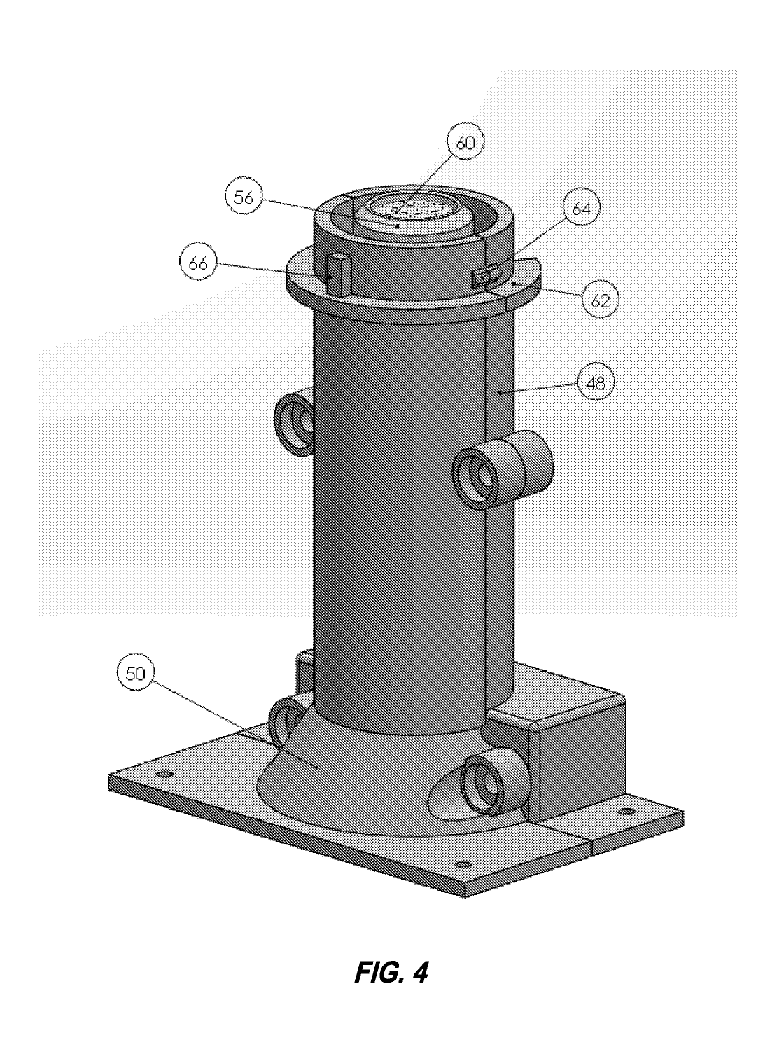

FIG. 4 is a perspective view of an example of a convective heating column with a bowl unit clipped to the heating column. FIG. 4 shows enclosure 50, steel mesh cartridge cover 60, steel tube 56, fan side of enclosure 48, and smart clips 64 for attaching the bowl.

The top of the enclosure pair 48 & 50 is functionalized with a ledge 62 and smart clips 64, which allow the heating column to snap into the bottom of the bowl 20. The smart clips are simply small round ridges in the slightly flexible plastic that are counter parts for the bowl smart clips 72.

Finally, the wide base of the plastic enclosures A and B screw into the device's base 32, making the column 26 a stable platform for attaching the bowl 20, and filling head 18.

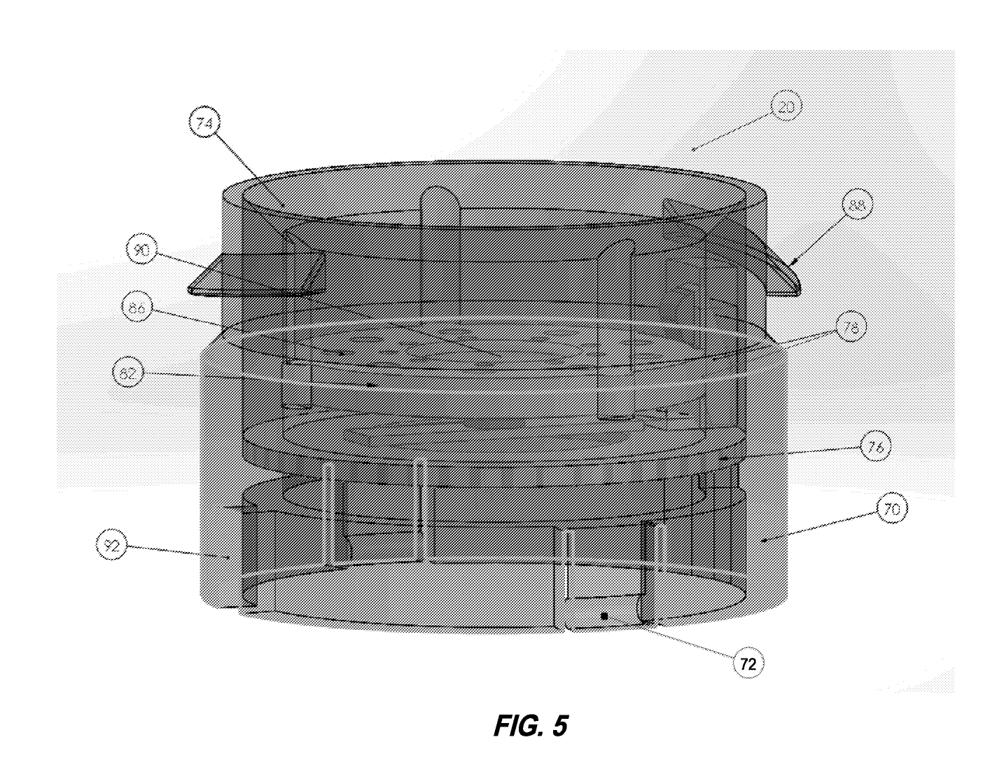

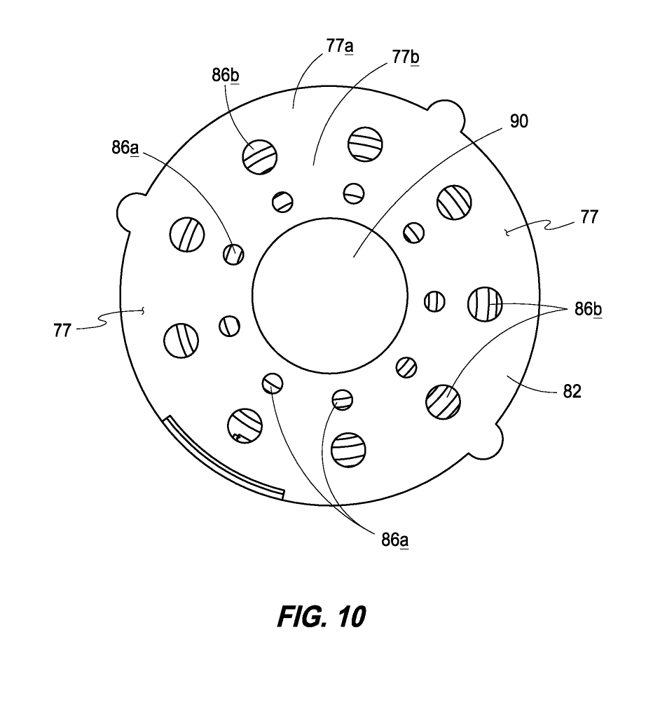

FIG. 5 is a perspective transparent view of an example of a material vaporization chamber or bowl 20 with a conductive heater 82 which may be utilized in embodiments of this invention. FIG. 5 illustrates a material vaporization surface 77 on a convective heater 82, the material vaporization surface in this embodiment includes a central portion 90 which is configured and disposed to receive and retain liquid, extract or oil. The outer portion of the material vaporization surface 77 is configured with a plurality of convection air apertures 86, which may also be referred to as air diffusing holes. It can be seen better in FIG. 10 that the convection air apertures 86 include smaller apertures 86a toward the center portion 90 and larger convection air apertures 86b towards the outer portion, to reflect a configuration to achieve a more uniform air and heat flow through the herbal material portion (or outer portion) of the material vaporization surface 77.

In this embodiment, the material vaporization surface 77 is part of a conductive heater 82 which includes heating coils (shown in other figures) which are disposed to provide heat to the material vaporization surface 77. FIG. 5 further illustrates the bowl cartridge adapter plate 76 to adapt the bowl or vaporization chamber to operably attach to the convective heater cartridge (shown as item 58 in other figures).

FIG. 5 further illustrates outer bowl shell 70, bowl inner housing 74 (preferably made out of steel), bowl smart clips 72, silicon electrode holder 80, bowl electrodes 78 and a locking mechanism 92 for attaching the column.

In the operation of some embodiments of this invention, the herbal material will be slightly ground and sit directly on top of the material vaporization surface 77, diffusing conductive heater 82, while fluid may be applied in small amounts (a drop or two) directly onto the indentation for oil/extract 90 in the middle of the diffusing conductive heater 82. The diffusing conductive heater may be utilized when vaporizing both herbal material and oil, as described elsewhere herein.

In some embodiments of this invention, the source of electrical energy to provide the heat may be provided by a nickel chromium (′nichrome′ resistive heating wire) coil 84 fused to the underside of the plate 82. The diffusing conductive heater plate may be made of a ceramic material so that it does not conduct electricity, assuring that the electrical current flows through the heating wire and not the plate. The plate should be thermally conductive in order to efficiently transmit or provide heat to the herbal material or oil on the material vaporization surface. Possible materials suitable for the plate material or material vaporization surface may be aluminum oxide or ceramics.

The material vaporization surface 77 of the conductive heater may include convection air apertures 86 in it to allow for air flow from the convective heating column 26. The holes also serve to diffuse the hot air from the convective heater as it passes into the bowl's inner chamber. This furthers an objective that the herbal material be vaporized more uniformly, rather than vaporizing only the herbal material closest to the center of the chamber (which may be typically hotter than the outer portions in this embodiment). This is one of a number of possible configurations and sizes of convection air apertures, with no one in particular being required to practice this invention.

The diffusing conductive heater 82 includes ridges that slide into small grooves on the bowl's steel inner housing 74. This small and relatively inexpensive part can be removed from the bowl entirely and cleaned or replaced if the part wears out.

The bowl can also easily or readily be removed from the convective heating column for convenient exchange of herbal material or fluid. When the bowl or vaporization chamber is attached to the convective air column, however, it may be secured by complementary features on the outer bowl shell 70 and the tops of the enclosures A and B 48 & 50. The lock on the bowl 92 fits over a key ridge on the enclosures 66, ensuring that the bowl will be oriented at the correct, fixed angle or radial location. This is utilized so that the correct electrodes on the bowl contact their counterparts on enclosure A 48; and also so that the bowl smart clips 72 fit correctly over enclosure A's smart clips 48.

The bowl smart clips 72 fit over the lower clips 64 on enclosures A and B (items 48 & 50) when the user gently pushes the bowl downward onto the heating column 26. The clips are offset so that they fit almost over each other, but not quite. This produces a slight tension that gently pulls the bowl downward, keeping it tightly attached to the heating column.

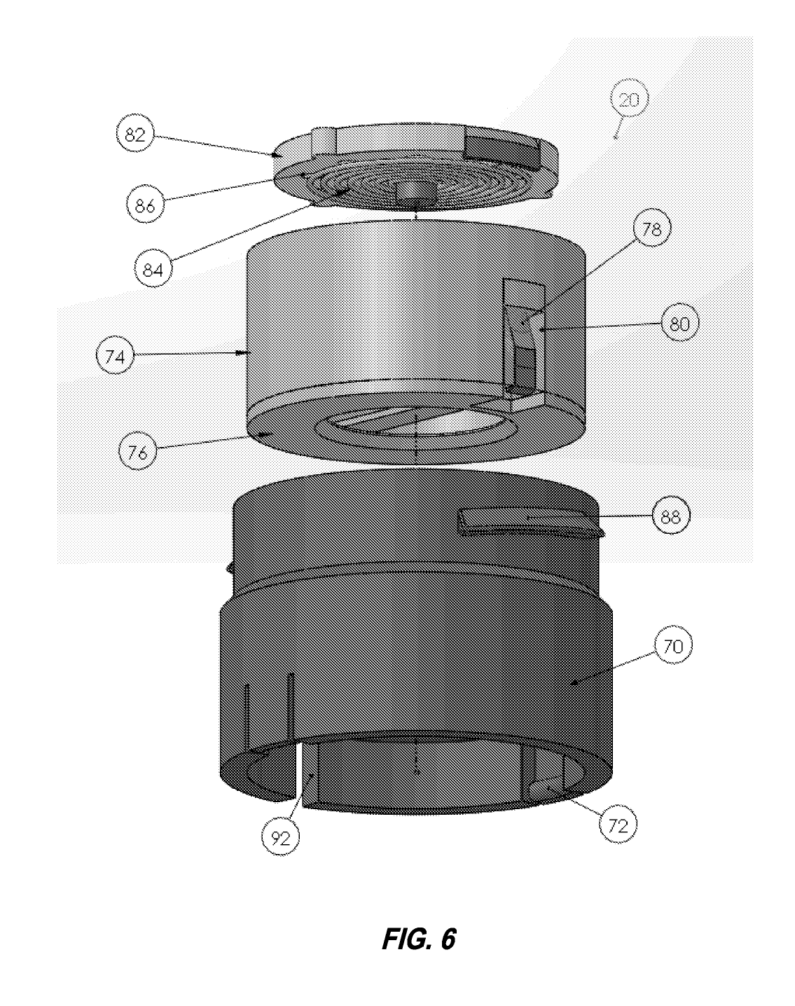

FIG. 6 is an elevation perspective exploded view of an example of a vaporization chamber or bowl unit 20, showing conductive heater 82 with material vaporization surface, that may be utilized in embodiments of this invention. FIG. 6 illustrates steel inner framework 74, bowl cartridge adapter plate 76, bowl electrodes 78, silicon electrode holder 80, outer bowl shell 70, screw clamps for filling head 88, smart clips 72 for the bowl and a lock for column attachment 92. The conductive heater 82 is shown with convection air apertures 86, a conductive heating coil 84 operably attached to and integral with the convective heater 82.

Electrical components in the bowl are best shown in an exploded view in FIG. 6. When the bowl is attached, electricity flows from the printed circuit board 36 up the wires 68 to a positive electrode contact 78. An outer electrode on the diffusing conductive heater 82 picks up electricity from this bowl electrode and transmits it to the nichrome coil. The current flows from the outside of the coil to the center, where a second electrode is fused to the conductive heater. This electrode connects downward to the metal bridge in the center of the bowl-cartridge adapter plate 76. This metal plate carries the current back to the negative electrode, where it returns down another wire to the PCB. The positive electrode may be insulated from the metal plate (and thus, the negative electrode) such as by a soft silicon insert.

The top of the outer bowl shell is functionalized to allow for the air-tight fastening of the filling head 18 onto the top of the bowl. The bowl's steel inner housing 74 may be chamfered to fit the chamfered edge of the filling head's inner steel chamber 102. The filling head lowers onto the top of the bowl and the complementary screw ledges 88 & 106 are used to lightly screw-clamp the head onto the bowl, sealing the air chamber for the hot air to flow through.

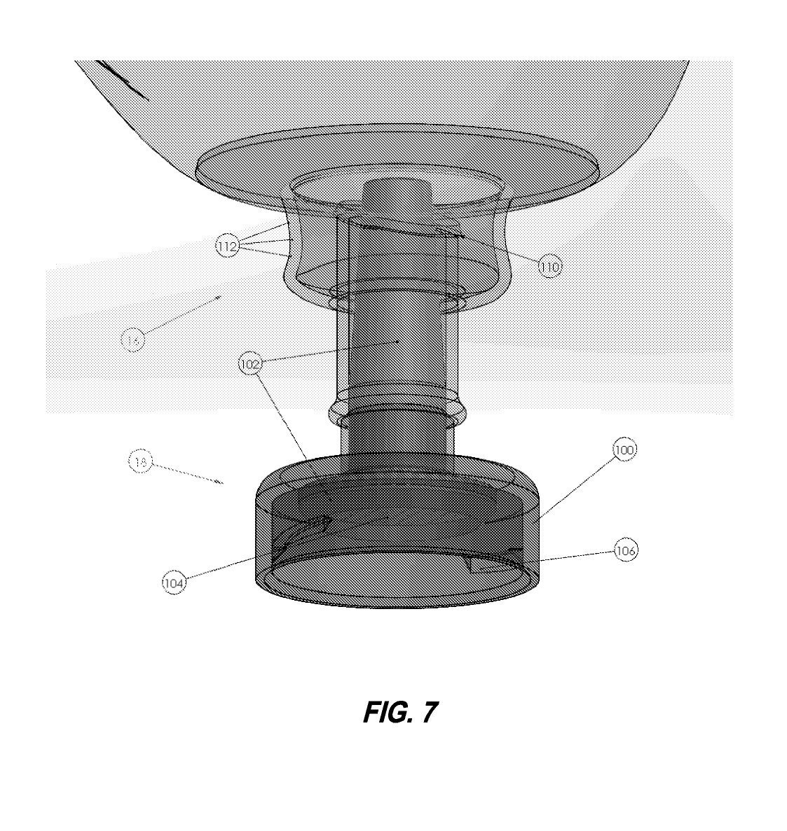

FIG. 7 is a perspective view of an example of a container filling adapter or filling head 18 which may be utilized in embodiments of this invention. FIG. 7 illustrates a containment bag mouthpiece 16, inner steel chamber 102, plastic outer cap 100, filling head screen 104, self-closing bag spout 110, and ergonomic dimples for the spout opening 112.

Filling head 18, vapor output, and self-closing bag: FIG. 7 shows the constituent parts of the filling head. The inner steel chamber 102 of the filling head is a machined (CNC) part that is bonded to the plastic outer cap 100. It is chamfered around the edge to fit into the bowl's steel inner 74 chamfer, as previously described. In the inner lip of the inner steel chamber 102 is a small circular groove that a metal screen can be popped into. This screen prevents herbal material passing with the flowing air out of the chamber and into the bag or hose. Vapor from herb/fluid in the bowl, however, flows up the inner chamber's spout, and into a bag or hose, depending on which the user is using.

In the embodiment of the invention where a containment bag is not utilized but instead a hose is directly attached to the adapter and the user then draws the vapor out of the bowl. The inner chamber's spout is tapered, such that it will be easy to slide the base end of the hose onto the spout. The base of the spout, however, has a diameter slightly larger than the inner diameter of the hose. Consequently, the hose can be slightly stretched down onto the spout, and the resultant friction will easily hold the hose in place. Here, air flow is induced by the user inhaling from the hose and the fan is not active.

The same filling head may be used to fill bags with vapor. When filling bags, the bag mouthpiece 16 rather than the hose end is pulled down over the inner chamber spout 102. Again, the bag spout's inner diameter is slightly smaller than the chamber outer diameter, so that friction holds the bag in place during filling. The soft plastic mouthpiece 16 has two features molded into its shape that allow for convenient filling of the bag and inhalation of vapor from the bag. The self-closing inner spout 110 is simply a cap that keeps vapor from escaping when at rest. The spout is slit directly underneath the cap, however, so that the cap can be bent away by applying pressure on its sides. Ergonomic press dimples 112 are molded into the side of the mouthpiece part so that users know exactly where to press on the inner spout 110 to easily open it. Thus, users can easily open the bag to inhale, but it will automatically close as soon as pressure is released, preventing vapor from being wasted. This same cap in 110 gets pushed back by the spout of the inner steel chamber 102 when the whole mouthpiece 16 is mounted. This opens the air path so that the bag can be filled as long as it's attached to the filling head.

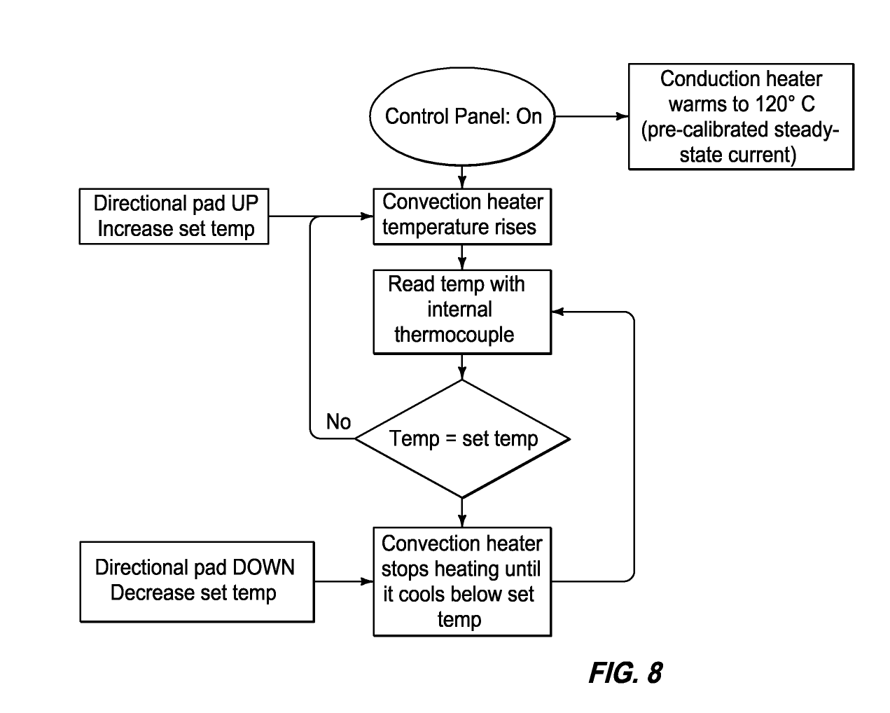

The device may operate in at least two distinct modes, which determine the functioning of the two heaters 58, 82. The two primary modes, “Herb Mode” and “Extract Mode”, are toggled between via the control panel 30 and are optimized for efficient vaporizing of either herb material or fluid extract. Extract Mode is the simpler of the two modes, and a flowchart is presented in FIG. 8 to illustrate how the device functions in Herb Mode.

When vaporizing fluid extract, convective heating (hot air flow from 58) is not as efficient as conductive heating. Poor air contact of the fluid in the center of the diffusing conductive heater plate 82 will cause vaporization to take a longer than desirable amount of time. Accordingly, in embodiments of this invention the convective heater is not used in Extract Mode. Instead when the device is in Extract Mode, the fluid is vaporized by direct, conductive heating from the heater plate 82, operating at around 200° C.; a temperature sufficiently beyond the flash point of common extract fluids. This may be accomplished by running a constant current through the nichrome coil 84 that is pre-calibrated to achieve that temperature. Time to heat the low-mass ceramic plate (choose ceramic with high thermal conductivity) and efficiently volatilize a small drop of fluid will be short.

In Herb Mode, the convective heater 58 will be the primary heater; however, the presence of the second conductive heater may offer a great additional benefit for vaporizing herbal material. Relying only on convection to initially warm up herbal material to vaporization temperatures can be highly inefficient, typically taking up to ten minutes, or requiring extended air flow before any vapor is produced. In Herb Mode, the conductive heater 82 may be employed at a steady lower temperature (˜100 C), directly warming the herb to slightly below the vaporization temperature of the targeted substance in the herbal material. This way, the additional flow of hot air (likely about 150 degrees Celsius—but which may be set by the user) will still vaporize the herb smoothly, but users will be able to reach the vaporization temperature with much lower waits.

The control panel 30 shown in FIG. 1 allows the user to control all relevant functions of the device. The left, round button turns Herb Mode on and off, while the diamond shaped button on the left turns Extract Mode on and off. In Herb Mode, the up/down buttons on the directional pad change the set temperature of the heating cartridge 58. In either Herb or Extract Mode, the fan can be employed to fill bags, and it is activated by the left/right arrows on the directional pad. The right button turns the fan on and up, while the left button turns the fan down and off. The fan has 3 speeds, so left/right navigate the following spectrum: off [symbol] 1 [symbol] 2 [symbol] 3.

The LCD displays the set temperature on top, with the live cartridge temperature displayed on bottom. Droplet and leaf icons also alternate to indicate whether the device is operating in Herb or Extract Mode.

Modularity. An additional feature in some embodiments of this invention includes a modular design and configuration which allows for readily replaceable components and subcomponents of the invention. For example the bowl 20 or vaporization chamber easily fits onto heating column 26 such that it can be readily removed and replaced. Another example is that the diffusing conductive heater plate 82 (which will tend to wear the quickest), is a relatively small and inexpensive component to replace, and fits easily into the bowl. Other examples are how the filling head 18 screws onto the bowl, and the bags, which also must be replaced periodically, fit onto the filling head.

The heater may also include a temperature sensor and feedback mechanism to monitor, control, maintain and/or regulate the temperature of the hot air and of the conductive heat supply. For example the convective heater cartridge may include an internal thermocouple.

The outer dome shape of the device also has innovative benefits. Similar previous devices have inner components arranged vertically, requiring a columnar shape with a high center of gravity. Arranging the fan to the side of the heating column 26, and wicking air up with the curved cavity in enclosures A and B 48 & 50 allows the overall height of the device to be much shorter. The low center of gravity makes the device very stable from being knocked over or pulled over via the hose. Air vents 46 were also positioned on the back of the device rather than the bottom, so that the bottom could be coated with soft silicon 34 and rest directly on a surface, providing further stability. The dome shape is also the most geometrically robust structure, protecting the device from crushing and impact forces.

The device has a battery harness 44 and a connection on the PCB 36 that allows the user to plug in an optional battery 42, providing portability. The PCB, featured in FIG. 2, is enclosed by a protective housing 38 that fits flush within the housing shell 12.

The side of the housing shell 12 has molded into it a drawer compartment, providing a storage function at virtually no extra manufacturing cost. A small plastic side door cover 24 snaps in to cover the drawer.

Smart clips 64 in various plastic components allow for tight fastening of parts without the need for screws or other parts.

FIG. 8 is an example of one flowchart of control and electronic functions for the herb material mode, which may be utilized in embodiments of this invention. When the unit is turned on, the conduction heater begins to warm to a temperature of approximately 120 degrees C., which may be pre-calibrated based on the current and control configuration of conduction heater and the control panel. At the same time the convection heater temperature begins to rise. The temperature to which the convection heater rises is based on the direction pad on the control panel.

As the temperature rises the internal temperature is measured by an internal thermocouple, and this information is provided back to the control panel to compare the temperature sensed with the temperature setting input by the user at the control panel. If the temperature sensed by the thermocouple is not yet up to the temperature input by the user, the electricity provided to the convection heater is continued and the process is continued until the set temperature is reached.

Once the set temperature is reached, the convection heater stops heating until it cools below the set temperature. The convection heater can also stop heating when the user re-sets the temperature at the control panel with the directional arrows, such as by engaging the down arrow to lower the set temperature, all as shown in FIG. 8.

For convenience, an inexpensive air flow sensor will be mounted at the base of the air flow chamber and integrated with the electronics. This will be used to sense when the bag is filled, signaling the fan to automatically turn off. In Herb Mode, the conductive heater 82 can also be signaled to increase its temperature when air flow is detected, when a user is inhaling from the hose, for example.

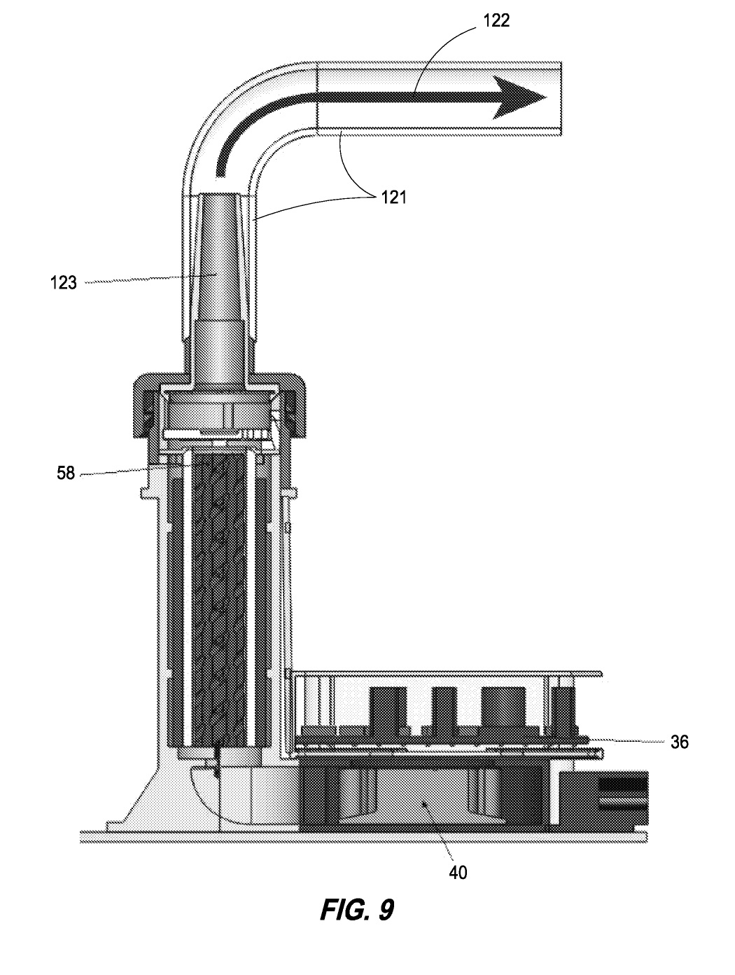

FIG. 9 is an elevation cross-sectional view of an example of an embodiment in which a hose 121 is attached as a conduit for a user to draw or inhale the air 122 containing the vaporized material. FIG. 9 illustrates printed circuit board 36, fan 40, convective heater cartridge 58 and air outlet spout 123.

FIG. 10 is a top view of an embodiment of the conduction heater 82 and material vaporization surface 77. FIG. 10 illustrates a material vaporization surface 77 on a convective heater 82, the material vaporization surface 77 in this embodiment includes a central portion 90 which is configured and disposed to receive and retain liquid, extract or oil. The outer portion of the material vaporization surface 77 is configured with a plurality of convection air apertures 86, which may also be referred to as air diffusing holes. It can be seen that the convection air apertures 86 include smaller apertures 86a toward the center portion 90 and larger convection air apertures 86b towards the outer portion, to reflect a configuration to achieve a more uniform air and heat flow through the herbal material portion (or outer portion) of the material vaporization surface 77.

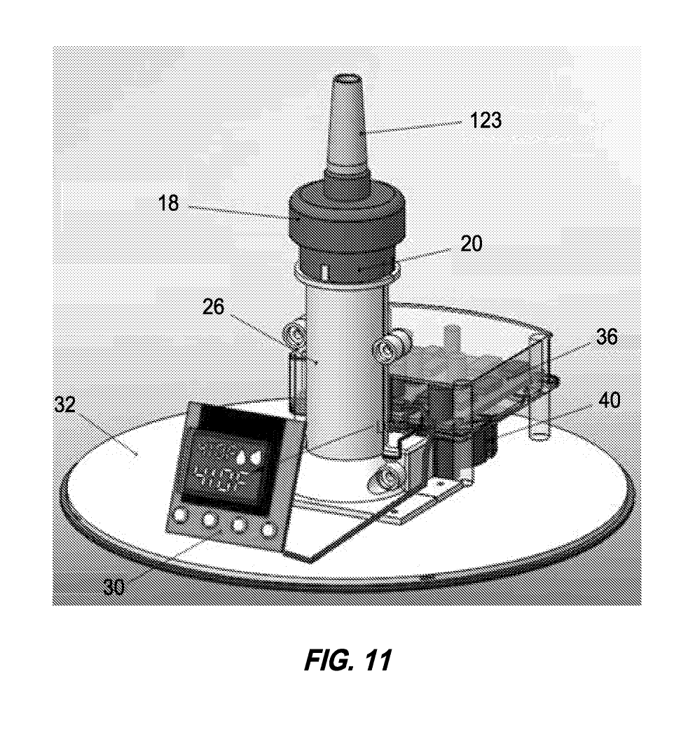

FIG. 11 is a perspective view of the embodiment of the invention illustrated in FIGS. 1 & 2 with the housing removed, illustrating base 32, control unit 30, convective heater cartridge 26, filling head 18, fan 40, printed circuit board 36, and vaporization chamber or bowl 20, wherein the base 32 in this example is part of the framework.

As will be appreciated by those of reasonable skill in the art, there are numerous embodiments to this invention, and variations of elements and components which may be used, all within the scope of this invention. In one embodiment for example, vaporizer system is provided for vaporizing herbal material and/or fluid material for eventual inhalation, comprising: a framework; a convective heat source mounted to the framework; a material vaporization surface configured to support material to be vaporized and to receive heated air from the convective heat source, the material vaporization surface including a plurality of convective air flow apertures through which the heated air from the convective heat source flows; a conductive heat source disposed to provide energy to heat the material vaporization surface; an air flow conduit disposed to receive air containing vaporized material from the material vaporization surface, and routing said air for further use.

In addition to the embodiment disclosed in the preceding paragraph, the invention may further include such a vaporizer system wherein: the system further comprises an outer housing attached to the framework; wherein the material vaporization surface has a liquid material vaporization portion and an herbal material vaporization portion, with the herbal material vaporization portion including the convective air flow apertures and the liquid material vaporization portion being disposed to contain the liquid material; wherein the material vaporization surface is comprised of a liquid material vaporization piece separate from an herbal material vaporization piece; further wherein the material vaporization surface is one of a screen, a mesh or an interwoven material which allows convective air flow through air flow apertures therein; and/or further comprising a control system mounted to the framework and operably attached to the convective heat source and the conductive heat source.

In addition, further embodiments of the control system may be wherein: the control system is configured such that the system may be operated solely in convective heat source mode or conductive heat source mode; the control system is configured such that the system may be operated in a mode that utilizes both the convective heater and the conductive heat source; and/or wherein the control system is configured to activate only the conductive heat source only when fluid material is being vaporized.

Still further embodiments of the vaporizer system recited in the second preceding paragraph may be: further wherein the convective heat source is one of forced convection or actively drawn convection; wherein the system further comprises an air flow fan disposed relative to the convective heat source to flow heated air from the convective heat source to the material vaporization surface; further wherein the material to be vaporized is one of herbal material or fluid material.

In a still further embodiment, a vaporizer system may be provided further wherein the material vaporization surface includes center portion convective air flow apertures and outer portion convective air flow apertures, sized such that the center portion convective air flow have an aperture cross-sectional area less than the outer portion convective air flow apertures

In compliance with the statute, the invention has been described in language more or less specific as to structural and methodical features. It is to be understood, however, that the invention is not limited to the specific features shown and described, since the means herein disclosed comprise preferred forms of putting the invention into effect. The invention is, therefore, claimed in any of its forms or modifications within the proper scope of the appended claims appropriately interpreted in accordance with the doctrine of equivalents.

I/We claim:

1. A vaporizer system for vaporizing herbal material and/or fluid material for eventual inhalation, comprising:
a convective heat source;
a material vaporization surface configured to support material to be vaporized and to receive heated air from the convective heat source, the material vaporization surface including a plurality of convective air flow apertures through which the heated air from the convective heat source flows;
a conductive heat source disposed to provide energy to heat the material vaporization surface;
an air flow conduit disposed to receive air containing vaporized material from the material vaporization surface, and routing said air for further use.

2. A vaporizer system as recited in claim 1, and further comprising a framework to which the convective heater is attached.

3. A vaporizer system as recited in claim 1, and further wherein the material vaporization surface has a liquid material vaporization portion and an herbal material vaporization portion, with the herbal material vaporization portion including the convective air flow apertures and the liquid material vaporization portion being disposed to contain the liquid material.

4. A vaporizer system as recited in claim 1, and wherein the material vaporization surface is comprised of a liquid material vaporization piece separate from an herbal material vaporization piece.

5. A vaporizer system as recited in claim 1, and further wherein the material vaporization surface is one of a screen, a mesh or an interwoven material which allows convective air flow through air flow apertures therein.

6. A vaporizer system as recited in claim 1, and which further comprises a control system mounted to the framework and operably attached to the convective heat source and the conductive heat source.

7. A vaporizer system as recited in claim 6, and further wherein the control system is configured such that the system may be operated solely in convective heat source mode or conductive heat source mode.

8. A vaporizer system as recited in claim 6, and further wherein the control system is configured such that the system may be operated in a mode that utilizes both the convective heater and the conductive heat source.

9. A vaporizer system as recited in claim 6, and further wherein the control system is configured to activate only the conductive heat source only when fluid material is being vaporized.

10. A vaporizer system as recited in claim 1, and further wherein the convective heat source is one of forced convection or actively drawn convection.

11. A vaporizer system as recited in claim 10, and further comprising an air flow fan disposed relative to the convective heat source to flow heated air from the convective heat source to the material vaporization surface.

12. A vaporizer system as recited in claim 10, and further wherein the material to be vaporized is one of herbal material or fluid material.

13. A vaporizer system as recited in claim 10, and further wherein the material vaporization surface is located vertically above the convective heat source.

14. A vaporizer system as recited in claim 13, and further wherein the material vaporization surface is located directly and not tangentially above the convective heat source.

15. A vaporizer system as recited in claim 1, and further wherein the convective air flow apertures are sized and configured to seek uniformity in air flow from the convective heat source through the convective air flow apertures in the material vaporization surface.

16. A vaporizer system as recited in claim 15, and further wherein the material vaporization surface includes center portion convective air flow apertures and outer portion convective air flow apertures, sized such that the center portion convective air flow have an aperture cross-sectional area less than the outer portion convective air flow apertures

17. A vaporizer system as recited in claim 1, and further wherein the conductive heater is a diffusing conductive heater.

18. A vaporizer system as recited in claim 2, and further comprising an outer housing attached to the framework.

19. A vaporizer system as recited in claim 2, and further wherein the framework is a base.
